(19) United States
(12) Patent Application Publication
(10) Pub. No.: US 2004/0038266 A1
(43) Pub. Date: Feb. 26, 2004

(54) Advancing the detection of hearing loss in newborns through parallel genetic analysis

(76) Inventors: Steven F. Dobrowolski, Park City, UT (US); Zhili Lin, Pittsburgh, PA (US)

Correspondence Address: MCKAY & ASSOCIATES, PC., 801 MCNEILLY ROAD, PITTSBURG, PA, 15226, US

(21) Appl. No.: 10/443,545

(22) Filed: May 22, 2003

Related U.S. Application Data
(60) Provisional application No. 60/370,762, filed on May 28, 2002.

(51) Int. Cl. C12Q 1/68; C12P 19/34

(52) U.S. Cl. 435/6; 435/91.2

(57) ABSTRACT

A newborn screening method is provided for detecting the causes of hereditary hearing loss. Patient specimen amplicons are synthesized, wherein the amplicon is an oligonucleotide specific to a gene selected from the group consisting of cytomegalovirus (CMV), mitochondria, and connexin 26 (Cx26). They are then spotted on a substrate and immobilized as a target for microarray production as wild type and mutated alleles are allowed to hybridize thereto and undergo image analysis.

### SPECIFIC REFERENCE

This application hereby claims benefit of provisional application serial No. 60/370,762, having a filing date of May 28, 2002.

### GOVERNMENT RIGHTS

This invention was made with the United States Government support under Grant No. 1R44DC04902-01 from the NIH. The United States Government has certain rights in this invention.

### BACKGROUND

Profound sensorineural hearing loss affects at least 0.1% of the general newborn population, while an equal number are found to have lesser, but clinically significant degrees of hearing loss. This frequency is far higher than observed in disorders, such as PKU and congenital hypothyroidism, that are routinely screened for in the United States and other countries. It is now clearly established that auditory stimulation in the first six months of life is required to drive the development of normal speech and language. Sensory deprivation, due to hearing impairment, inhibits this development resulting in learning dysfunction, along with impaired social and emotional development.

Screening newborns for hearing loss using auditory methods is generally performed using a two tiered approach. In the first tier, newborns are screened using transient evoked otoacoustic emission (TEOAC). TEOAC measures sounds generated by the hair cells in the cochlea in response to acoustic stimulation. The sounds generated are indicative of the integrity of the inner ear. Those who fail TEOAC are referred for second tier audiometric testing using the more sensitive auditory brainstem response (ABR) assay. ABR is considered the standard for assessment of hearing in neonates and infants by measuring electroencephalographic waveforms in response to clicks. ABR assesses the outer, middle, and inner ear but also lower auditory pathways. The 2-tiered approach (TEOAC followed by ABR) to screening newborns for hearing loss via audiometric means is effective, however major caveats exist that reduce its overall sensitivity and specificity.

The logistics, associated with two-tiered auditory screening, results in many newborns being lost to follow-up. Newborns failing the TEOAC assay are required to return at a later date for ABR analysis. Studies have also indicated that 10-15% of newborns do not receive the first tier TEOAC assay due to early discharge and/or lack of tester time. While auditory testing is effective, short comings exist (e,g. test availability, logistics of 2^{nd} tier ABR analysis, sensitivity/specificity issues) and there is a clear need to improve the overall process. Owing to the widespread observance of common genetic abnormalities in connexin 26 gene and genes of mitochondrial origin, parallel genetic analysis holds great potential to improve screening for hearing loss. Screening newborns using molecular means to supplement auditory screening will provide critical data to confirm diagnosis of deafness in affected infants. Additionally, molecular genetic analysis has the added benefit of being able to identify infants who are at risk for late onset hearing loss that are currently missed by current audiologic screening programs.

Some infectious agents are responsible for hearing loss and the most common of these infectious agents that may cause hearing loss is cytomegalovirus. In the United States, approximately 1% of newborns are congenitally infected with the cytomegalovirus (CMV). A small minority of these infected newborns (10%) are symptomatic at birth (e.g. intrauterine growth retardation, hepatosplenogegaly, thrombocytopenia, intracranial calcifications), while the vast majority go unrecognized or diagnosis is delayed. Hearing loss attributable to CMV may be present at birth or develop postnatally. The severity of CMV induced hearing loss may vary from moderate to profound. Hearing loss associated with infection by CMV could be prevented by screening newborns for congenital CMV infection.

The majority of hereditary hearing loss (70%) is nonsyndromic, wherein the inner ear is the only body system affected. These nonsyndromic forms of hearing loss are particularly suited to prospective newborn screening because there may be no other overt clue to the presence of a hearing disorder. There are many genes involved in nonsyndromic hearing loss however genes of mitochondrial origin and connexin 26 (Cx26) account for the majority of hereditary deafness. In 1997 it was reported that mutations involving the Cx26 gene were the cause of hereditary deafness, owing to the DFNB 1 loci. In generic terms connexin describes a family of gap junction proteins that code for the membrane bound protein sub-units that line intercellular pores connecting adjacent cells and facilitate the movement of small molecules. Cx26 is a small gene with no introns and single exon of 798 base pairs. Connexin 26 is expressed in the cochlea and may contribute to regulation of potassium concentration in the cochlear endolymph. At least 21 pathologic mutations have been described, but one, the 35delG accounts for up to 70% of the observed mutations in many populations. Other high prevalence mutations include the 167del T which has a carrier frequency of about 4% in Ashkinazi Jews, 235delc found in many Asians, R143W that initially identified in Japanese but also identified frequently in Africans, and M34T which is widely distributed among diverse populations.

Techniques for DNA analysis have crossed a threshold of technical proficiency enabling population wide screening utilizing molecular genetic methods. In fact, for many years, gene level analysis has been performed using traditional methods such as electrophoresis, sequencing, or hybridization with an oligonucleotide probe. These methods are effective, however they are labor intensive and poorly amenable to automation and high throughput. A recent development for gene level analysis is the microarray. Microarrays are devices where minute quantities of DNA molecules are immobilized to an underlying substrate. High-density DNA microarrays are routinely fabricated with an excess of 100,000 distinct elements (synthetic DNA, PCR product, cDNA, etc) immobilized to defined sites in an array. Each site in the array identifies a unique genetic feature. Microarrays decipher complex gene expression patterns or analyze numerous diagnostic amplification products in a single assay. There is a need then for a newborn screening method for detecting the causes of hereditary hearing loss utilizing microarray technology and an oligonucleotide panel specific for hereditary hearing loss.

### SUMMARY OF THE INVENTION

Accordingly, what is provided is a genetic screening method to detect the most common causes of hereditary hearing loss comprising the steps of synthesizing (using polymerase chain reaction) a patient specimen amplicon, wherein the amplicon consists of a section of the selected gene and contains the genetic site of interest; printing the amplicon on a substrate, thereby forming an immobilized target; querying the immobilized targets for genotype assignment by hybridization to a fluorescent-labeled probe, wherein the probe (defined by nucleotide sequence) is specific to a wild type or mutant allele complement to the target; thereby forming a hybridized microarray; scanning the microarray, thereby forming an image file of illuminated spots; and analyzing the image file by visual detection of and/or measurement of the amount and color of florescence in each spot. Through analysis of fluorescence intensity and color, the genotype of the specimen at those genetic sites of interest is determined.

### BRIEF DESCRIPTION OF THE DRAWING

The patent or application file contains at least one drawing executed in color. Copies of this patent or patent application publication with color drawing(s) will be provided by the Office upon request and payment of the necessary fee.

FIG. 1 shows an example of a colored image file produced by the present invention.

### DETAILED DESCRIPTION OF THE INVENTION

A “primer” is a short piece of synthetic single strand DNA complementary to a given DNA sequence and which acts as the initiation point from which replication proceeds via DNA polymerase.

“PCR” (polymerase chain reaction) as used herein and as generally known in the art is the rapid technique for amplification of a DNA or RNA sequence, wherein the oligonucleotide primers are annealed (form complimentary base pairing) to single stranded nucleotide sequences, which are copied by DNA polymerase replication.

“Hybridization” defines the process for annealing the complementary sequence through base pairing interaction between a probe and template.

“Target” is the tethered amplicon immobilized to the glass substrate of the microarray that is then available to accept the probe (complementary strand) through hybridization.

“Amplicons” refer to any DNA sequence prepared using polymerase chain reaction.

“Microarray” as defined herein is a specialized glass substrate to which amplicons are immobilized and as such are available to be hybridized with labeled probes and undergo image analysis to reveal differences in hybridization patterns. These differences in hybridization patterns are used for genotype assignment.

The oligonucleotide panel described herein includes strands of cytomegalovirus (CMV), connexin 26 (Cx26), and mitochondrial DNA for providing a complete microarray protocol for detecting the causes of hereditary hearing loss owing to the commonly observed genetic abnormalities occurring in these genes.

Connexins are a family of gap junction proteins that code for the membrane bound protein subunits that line intercellular pores connecting adjacent cells and facilitates the movement of small molecules. Cx26 is a small gene with no introns and a single exon of 798 base pairs. The sequence of the connexin 26 gene is shown as SEQUENCE ID NO: 1.

In the present invention, multiple mutations of the Cx26 gene are described as being indicators for hereditary hearing loss and are detected by using primers to amplify more than one designated portion of the gene. These gene fragments are subsequently immobilized on a glass substrate where they are queried for genotype designation using complementary probes. In the preferred embodiment of the present invention, four mutations on the Cx26 gene, plus the mutations/strands described below, are adapted to be detected simultaneously by determining an amount of fluorescence revealed after an image scan takes place when the probes are allowed to hybridize with each target amplicon. The Cx26 mutations include: 35 del., Cx26 167 del., Cx26 235 del., and Cx26 M34T, wherein the M is methionine encoded by ATG and the T is the threonine encoded by ACG.

Mitochondria also contain RNA and DNA, the means of which they independently replicate and code for the synthesis of some proteins, and nonsyndromic hearing loss can be caused by mutations to mitochondrial genes. The human mitochondrial genome contains about 16,567 nucleotide pairs and a sequence of which is designated herein as SEQ ID NO: 18. The multiple mutation panel as described herein includes the two mitochondrial mutations A1555G and A7445C. Mitochondrial mutations account for 8-25% of non-syndromic sensorineural hearing loss in some populations.

Cytomegalovirus (CMV) is an etiological agent that may cause hearing loss, completing the panel for hearing loss of the present invention. CMV is a member of the Herpesviridae family of large DNA viruses. Approximately 1% of all newborns are congenitally infected with CMV. Approximately half of the infants with symptomatic and 15% of infants with asymptomatic congenital CMV infection will present with hearing loss. A portion of a herpes virus sequence containing the relevant sequences that indicate the presence of CMV is set forth by SEQ ID NO: 27.

In accordance with the present invention, genomic DNA of known hereditary hearing loss (HHL) genotypes is extracted and purified. Genomic samples are collected from any traditional methods, such as from any tissue or organ from which RNA or DNA can be amplified, or by purification from a dried blood spot. In the current embodiment, DNA is extracted from a punch spot of a dried blood spot specimen retrieved from a filter paper card.

Primer design software is used to design the primers for the synthesis of patient specimen amplicons. Synthesis of the specimens is accomplished by PCR. In this embodiment, primers are designed such that Tm's fall within a 4.5° C. window centering around 61.0° C. This enables the use of common amplification conditions for all primer pairs. Table 1 below shows the primers developed for the current assay.

TABLE 1

| | | |
|---|---|---|
| Primer | Sequence | ID No. |

| | | | |
|---|---|---|---|
| Cx26 | Fwd | 5′ ATGGATTGGGGCACGCTG 3′ | 2 |
| 35 Del. | | | |
| Cx26 | Rev | 5′ C6-CAATGCTGGTGGAGTGTTTGT 3′ | 3 |
| 35 Del. | | | |
| Cx26 | Fwd | 5′ CCGACTTTGTCTGCAACACC 3′ | 6 |
| 167 Del. | | | |
| Cx26 | Rev | 5′ C6-GTGATCGTAGCACACGTTCTTGC 3′ | 7 |
| 167 Del. | | | |
| Cx26 | Fwd | 5′ CCCCATCTCCCACATCCG 3′ | 10 |
| 235 Del. | | | |
| Cx26 | Rev | 5′ C6-CGCTGGGCTGGACACGAAG 3′ | 11 |
| 235 Del. | | | |
| Cx26 | Fwd | 5′ CCGTCCTCTTCATTTTTCGC 3′ | 14 |
| M34T | | | |
| Cx26 | Rev | 5′ C6-TCTCCCCACACCTCCTTTGC 3′ | 15 |
| M34T | | | |
| Mito. | Fwd | 5′ CCCCTACGCATTTATATAGAGGA 3′ | 19 |
| A1555G | | | |
| Mito. | Rev | 5′ C6-CGTCCAAGTGCACTTTCCAGTAC 3′ | 20 |
| A1555G | | | |
| Mito. | Fwd | 5′ CCCACCCTACCACACATTCG 3′ | 23 |
| A7445C | | | |
| Mito. | Rev | 5′ C6-GGGGGTTCGATTCCTTCCT 3′ | 24 |
| A7445C | | | |
| CMV | Fwd | 5′ TTTGTTGTAAATGGCCGAGAGA 3′ | 28 |
| CMV | Rev | 5′ C6-CAACGGCGCACCCTAGAG 3′ | 29 |

A C6 amino modifier is attached to the 5′ end of the primer. The modifier group enables the attachment and isolation of the specific strand to the glass substrate used for microarray printing. For each PCR primer set, one or both primers have the C6 amino modifier. By doing this, only the one strand of the amplicon will attach to the glass slide, the other strand will be washed away during the slide processing steps which are performed after printing. Selection of a particular strand acts to enhance the hybridization efficiency by eliminating binding competition between the complementary amplicon strand and the assay probe for the target.

The amplicon is then spotted on the glass substrate and readied for hybridization. Fluorescent dye-labeled oligonucleotide probes matched to either wild type or mutant alleles were designed as indicated in table 2 below.

[image]

Hybridization is the process of incubating the immobilized target DNA tethered on the glass substrate with the labeled probe DNA at a particular temperature. The fluorescent probe DNA will hybridize with the primer-amplified, target DNA and the amount of immobilized fluorescence can be determined by a scan.

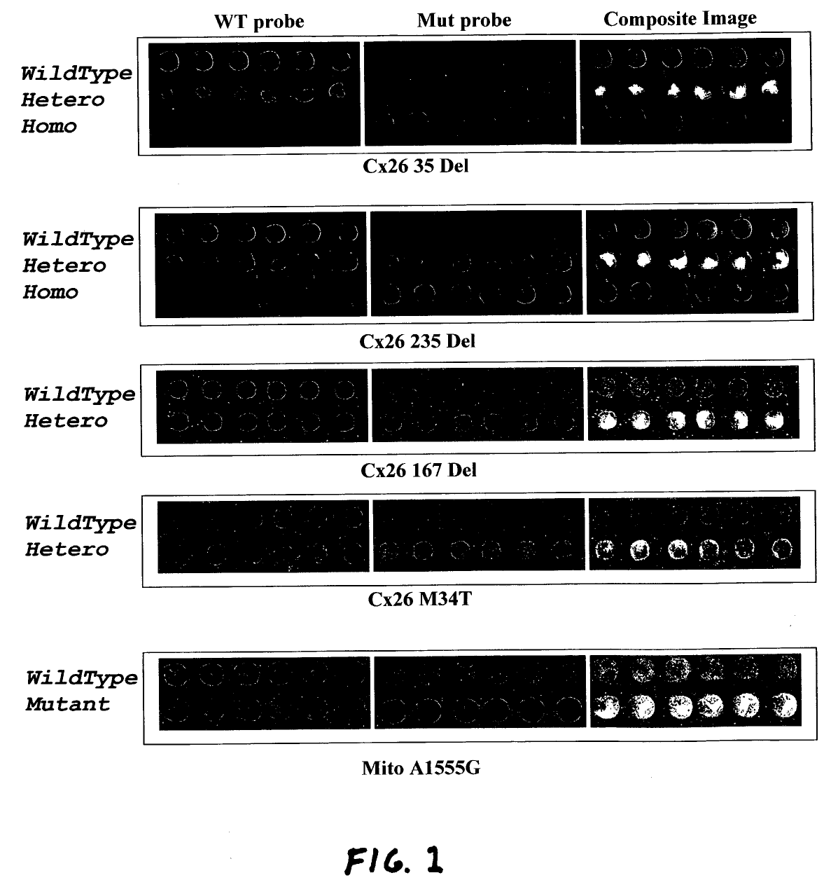

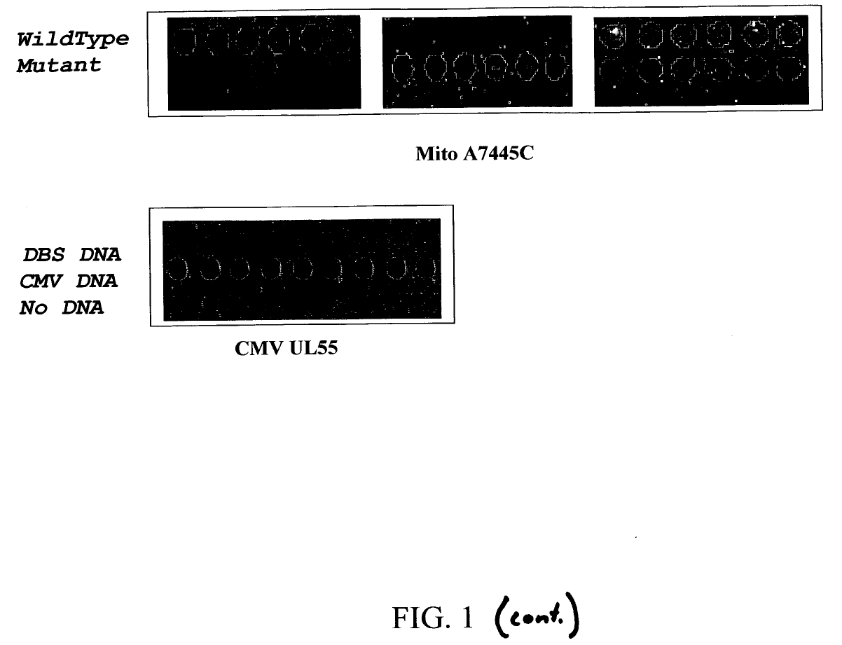

Each spot of DNA contains pixels, which are illuminated one pixel at a time using lasers until all the spots on the DNA chip have been scanned and recorded as a high-resolution image file. The scanned images are analyzed in an automated data extraction process that measures the absolute and relative fluorescence at two wavelengths. The use of the present targets and probes for DNA microarray analysis produces the hybridized microarray slide images as seen in FIG. 1, specific for detecting the causes of hereditary hearing loss.

### EXAMPLE

Samples and DNA Preparation

Genomic DNA of known HHL genotypes (Wild Type, Heterozygous, and Homozygous) were purified from Guthrie filter card Dried Blood Spots. DNA is extracted from a 3.2 mm punch of DBS.

PCR Amplification

Synthesis of patient specimen amplicons is accomplished by PCR. Primer Premier 5.0 and Oligo 6.0 are among the most advanced PCR primer design software packages and are both employed for primer design. Primers are designed such that Tm's fall within a 4.5° C. window centering around 61.0° C. This enables the use of common amplification conditions for all primer pairs.

All primers are synthesized and HPLC purified by Operon Technologies, Inc. (Alameda, Calif.). A C6 amino modifier is attached to the 5′ end of selected primers to enable the attachment and isolation of a specific strand to the glass substrate used for microarray printing. For each PCR primer set, only one primer has the C6 amino modifier. By doing this, only the one strand of the amplicon will be attached to the glass substrate, the other strand will be washed away during the processing step. Selection of a particular strand acts to enhance the hybridization efficiency by eliminating competition between the complementary amplicon strand and the assay probe for target binding.

The PCR amplification reaction (10 μl) contained 10 mM Tris-HCl; 1.5 mM MgCl₂; 50 mM KCl; 4 μl DNA; 0.5 μM each of primers; 200 μM each of dATP, dCTP, dGTP, and dTTP; 0.08 μg TaqStart Antibody (CloneTech); 0.4 unit Taq Polymerase (Roche). PCR was performed in a MWG Biotech PrimusHT Multibloack thermal cycler. Cycling condition are one cycle at 94° C. for 20 sec, 58° C. for 30 sec, 72° C. for 20 sec, and a final cycle at 72° C. for 2 min.

MicroArray Printing and Processing

There is no PCR purification needed after the PCR reactions are completed. Ten μl of sodium phosphate spotting buffer (300 mM sodium phosphate, pH 8.5/0.02% SDS) was added to each 10 ul PCR reaction, and then printed onto Eppendorf's CreativeChip Oligo slides(Hamburg, Germany) using Virtek Vision's ChipWriter Professional Arrayer (Waterloo, ON, Canada). Printed slides were incubated at 50° C. for 1 hour in a humidified chamber, baked at 80° C. for 1 hour, incubated with 200 ml boiled dH₂O for 3 min, then dried by centrifigation.

Hybridization

Fluorescent dye-labeled oligonucleotide probes matched to either wild type or mutant alleles were designed as indicated in table II, and synthesized. All probes were analyzed by spectrophotometry for oligonucleotide and fluorophore concentration. The Hybridization solution contains Quantifoil's QMT Hybridization buffer (Jena, Germany) with both wild type and mutant probes at a final concentration of 0.1 μM. Each hybridization reaction was carried out with 20 μl of hybridization solution added to the printed slides, covered with a glass coverslip, sealed in the hybridization cassette with 50 μl of dH₂O added to the humidity control chamber. The cassette was then put into a circulated water bath, incubated at 50° C. for 1 hour. After 1 hour incubation, the slide was taken out of the cassette and the coverslip was removed. Excess probe is immediately rinsed from the slide by dipping the slide into a room temperature solution of 2×SSC, 0.1% sarcosyl. Non-specific probe binding is then washed from the slide by placing the slide into a container of 2×SSC, 0.1% sarcosyl and incubated in this solution at 50° C. for 10′. The slides are then further washed by dipping 2 to 3 times in a room temperature solution of 2×SSC followed by dipping 2 to 3 times in a room temperature solution of 0.2×SSC. The slide is then immediately dried by centrifugation for 3′ at room temperature.

Microarray Scanning

Each hybridized microarray slide was scanned with Virtek Vision's ChipReader scanner. For both Cy3 and Cy5 channel of the scanner, laser power was set at 100, detector gain at 1, and the number of scans at 1, and detector sensitivity t 1000.

Image Composite

The two images from scanning of each microarray slide was composited with ArrayPro Analyzer (Media Cybernetics, L. P.)

Methods of Data Analysis and Interpretation

The resulting data may be analyzed using a variety of approaches. For the purposes of the present invention, two examples are herein described, though others may be readily apparent to those of ordinary skill in the art. The first approach is by visual inspection of the resulting wild type and mutant signal composite image as described below. Since the wild type and the mutant probes are labeled with different fluorophors, each will emit light at a different wavelength. When the slide is scanned at each of these two wavelengths, two images are created. One image corresponds to the wild type probe signal and the other image corresponds to the mutant probe signal. After the images are acquired, analysis software is used to assign each image a specific color. One color will be assigned to the signal resulting from the wild type probe and another color will be assigned to the signal resulting from the mutant probe. When the two images (wild type and mutant) are combined into a single composite image, all possible genotypes, including samples containing any combination of the mutant and wild type alleles (heterozygotes), can be visually detected. This visual detection is possible because samples containing a combination of wild type or mutant alleles will composite to produce additional computer generated colors which will differ from the colors originally assigned to the wild type and mutant alleles. One will only need to visually determine the sample image color to determine the sample genotype.

A second analysis approach is used to assign quantitative values to each genotype. In this approach, the slides are probed with both wild type and mutant probes of differing fluorophor emission wavelengths as in the above approach. Also as above, the slide is scanned and a separate image is created for the mutant signal and the wild type signal. From this point, the signal from each of the images is quantified using array analysis software. For each sample, a ratio of wild type to mutant signal is calculated. The resulting quantitative ratios can then be categorized into distinct value ranges for each of the possible genotypes. All of this quantification is performed by computer analysis, genotypes are computer assigned, and results are outputted for easy and rapid interpretation in a high-through-put screening laboratory.

I claim:

1. A genetic screening method for detecting a cause of hereditary hearing loss, comprising:
synthesizing a patient specimen amplicon, wherein said patient specimen amplicon comprises a section of a connexin 26 (Cx26) gene;
immobilizing said patient specimen amplicon onto a substrate, thereby forming target DNA; and,
allowing probes to hybridize with said target DNA, wherein said probes are selected from the group consisting of those sequences as set forth in SEQ ID NOS: 4, 5, 8, 9, 12, 13, 16, and 17, thereby forming a hybridized microarray slide specific for detecting mutations on said Cx26 gene.

2. The method of claim 1, further comprising the step of scanning said hybridized microarray slide to produce data for detecting an extent of hybridization of said probes with said target DNA.

3. The method of claim 2, wherein said data is color image data.

4. The method of claim 2, wherein said data is a quantitative ratio of a wild type to mutant signal.

5. The method of claim 1, wherein said patient specimen amplicon is synthesized from a dried blood spot on filter paper.

6. The method of claim 1, wherein for the step of synthesizing said patient specimen amplicon, a primer selected from the group of those sequences as set forth in SEQ ID NO: 2, 6, 10, and 14 is used as a forward primer.

7. The method of claim 1, wherein for the step of synthesizing said patient specimen amplicon, a primer selected from the group consisting of those sequences as set forth in SEQ ID NO: 3, 7, 11, and 15 is used as a reverse primer.

8. A genetic screening method for detecting a cause of hereditary hearing loss, comprising:
synthesizing a patient specimen amplicon, wherein said patient specimen amplicon comprises a section of a mitochondrial gene;
immobilizing said patient specimen amplicon onto a substrate, thereby forming target DNA; and,
allowing probes to hybridize with said target DNA, wherein said probes are selected from the group consisting of those sequences as set forth in SEQ ID NOS: 21, 22, 25, and 26, thereby forming a hybridized microarray slide specific for detecting mutations on said mitochondrial gene.

9. The method of claim 8, further comprising the step of scanning said hybridized microarray slide to produce data for detecting an extent of hybridization of said probes with said target DNA.

10. The method of claim 9, wherein said data is color image data.

11. The method of claim 9, wherein said data is a quantitative ratio of a wild type to mutant signal.

12. The method of claim 8, wherein said patient specimen amplicon is synthesized from a dried blood spot on filter paper.

13. The method of claim 8, wherein for the step of synthesizing said patient specimen amplicon, a primer selected from the group of those sequences as set forth in SEQ ID NO: 19 and 23 is used as a forward primer.

14. The method of claim 8, wherein for the step of synthesizing said patient specimen amplicon, a primer selected from the group consisting of those sequences as set forth in SEQ ID NO: 20 and 24 is used as a reverse primer.

15. A genetic screening method for detecting a cause of hereditary hearing loss, comprising:
synthesizing a patient specimen amplicon, wherein said patient specimen amplicon comprises a section of a cytomegalovirus (CMV) gene;
immobilizing said patient specimen amplicon onto a substrate, thereby forming target DNA; and,
allowing probes to hybridize with said target DNA, wherein said probe is that such sequence of SEQ ID NO: 30, thereby forming a hybridized microarray slide specific for detecting a presence of the CMV gene.

16. The method of claim 15, further comprising the step of scanning said hybridized microarray slide to produce data for detecting an extent of hybridization of said probes with said target DNA.

17. The method of claim 16, wherein said data is color image data.

18. The method of claim 16, wherein said data is a quantitative ratio of a wild type to mutant signal.

19. The method of claim 15, wherein said patient specimen amplicon is synthesized from a dried blood spot on filter paper.

20. The method of claim 15, wherein for the step of synthesizing said patient specimen amplicon, a primer as set forth by SEQ ID NO: 28 is used as a forward primer.

21. The method of claim 15, wherein for the step of synthesizing said patient specimen amplicon, a primer as set forth by SEQ ID NO: 29 is used as a reverse primer.


SEQUENCE LISTING (30 sequences)

SEQ ID NO: 1 - DNA, 2311 bp, Homo sapiens
gatttaatcctatgacaaactaagttggttctgtcttcacctgttttggtgaggttgtgtaagagttggtgtttgctcaggaagagatttaagcatgcttgcttacccagactcagagaagtctccctgttctgtcctagctatgttcctgtgttgtgtgcattcgtcttttccagagcaaaccgcccagagtagaagatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccagcccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaacgcattgcccagttgttagattaagaaatagacagcatgagagggatgaggcaacccgtgctcagctgtcaaggctcagtcgccagcatttcccaacacaaagattctgaccttaaatgcaaccatttgaaacccctgtaggcctcaggtgaaactccagatgccacaatgagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagagaggctgtctgttgtattcattgtggtcatagcacctaacaacattgtagcctcaatcgagtgagacagactagaagttcctagttggcttatgatagcaaatggcctcatgtcaaatattagatgtaattttgtgtaagaaatacagactggatgtaccaccaactactacctgtaatgacaggcctgtccaacacatctcccttttccatgctgtggtagccagcatcggaaagaacgctgatttaaagaggtgagcttgggaattttattgacacagtaccatttaatggggagacaaaaatgggggccaggggagggagaagtttctgtcgttaaaaacgagtttggaaagactggactctaaattctgttgattaaagatgagctttgtctaccttcaaaagtttgtttggcttacccccttcagcctccaattttttaagtgaaaatataactaataacatgtgaaaagaatagaagctaaggtttagataaatattgagcagatctataggaagattgaacctgaatattgccattatgcttgacatggtttccaaaaaatggtactccacatacttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataat

SEQ ID NO: 2 - DNA, 18 bp, Homo sapiens
atggattggggcacgctg

SEQ ID NO: 3 - DNA, 21 bp, Homo sapiens
caatgctggtggagtgtttgt

SEQ ID NO: 4 - DNA, 15 bp, Homo sapiens
tcctggggggtgtga

SEQ ID NO: 5 - DNA, 15 bp, Homo sapiens
ttcctgggggtgtga

SEQ ID NO: 6 - DNA, 20 bp, Homo sapiens
ccgactttgtctgcaacacc

SEQ ID NO: 7 - DNA, 23 bp, Homo sapiens
grgatcgtagcacacgttcttgc

SEQ ID NO: 8 - DNA, 15 bp, Homo sapiens
acaccctgcagccag

SEQ ID NO: 9 - DNA, 15 bp, Homo sapiens
tacacccgcagccag

SEQ ID NO: 10 - DNA, 18 bp, Homo sapiens
ccccatctcccacatccg

SEQ ID NO: 11 - DNA, 19 bp, Homo sapiens
cgctgggctggacacgaag

SEQ ID NO: 12 - DNA, 15 bp, Homo sapiens
ggccctgcagctgat

SEQ ID NO: 13 - DNA, 15 bp, Homo sapiens
ggcctgcagctgatc

SEQ ID NO: 14 - DNA, 20 bp, Homo sapiens
ccgtcctcttcatttttcgc

SEQ ID NO: 15 - DNA, 20 bp, Homo sapiens
tctccccacacctcctttgc

SEQ ID NO: 16 - DNA, 16 bp, Homo sapiens
cgaggatcataatgcg

SEQ ID NO: 17 - DNA, 16 bp, Homo sapiens
cgcattacgatcctcg

SEQ ID NO: 18 - DNA, 16567 bp, Homo sapiens
  misc_feature (3040)..(3040): n is a, c, g, or t
gatcacaggtctatcaccctattaaccactcacgggagctctccatgcatttggtattttcgtctggggggtgtgcacgcgatagcattgcgagacgctggagccggagcaccctatgtcgcagtatctgtctttgattcctgcctcatcctattatttatcgcacctacgttcaatattacaggcgaacatacttactaaagtgtgttaattaattaatgcttgtaggacataataataacaattgatgtctgcacagccgctttccacacagacatcataacaaaaaatttccaccaaacccccccctccccccgcttctggccacagcacttaaacacatctctgccaaaccccaaaaacaaagaaccctaacaccagcctaaccagatttcaaattttatcttttggcggtatgcacttttaacagtcaccccccaactaacacattattttcccctcccactcccatactactaatctcatcaatacaacccccgcccatcctacccagcacacacaccgctgctaaccccataccccgaaccaaccaaaccccaaagacaccccccacagtttatgtagcttacctcctcaaagcaatacactgaaaatgtttagacgggctcacatcaccccataaacaaataggtttggtcctagcctttctattagctcttagtaagattacacatgcaagcatccccgttccagtgagttcaccctctaaatcaccacgatcaaaagggacaagcatcaagcacgcagcaatgcagctcaaaacgcttagcctagccacacccccacgggaaacagcagtgattaacctttagcaataaacgaaagtttaactaagctatactaaccccagggttggtcaatttcgtgccagccaccgcggtcacacgattaacccaagtcaatagaagccggcgtaaagagtgttttagatcaccccctccccaataaagctaaaactcacctgagttgtaaaaaactccagttgacacaaaatagactacgaaagtggctttaacatatctgaacacacaatagctaagacccaaactgggattagataccccactatgcttagccctaaacctcaacagttaaatcaacaaaactgctcgccagaacactacgagccacagcttaaaactcaaaggacctggcggtgcttcatatccctctagaggagcctgttctgtaatcgataaaccccgatcaacctcaccacctcttgctcagcctatataccgccatcttcagcaaaccctgatgaaggctacaaagtaagcgcaagtacccacgtaaagacgttaggtcaaggtgtagcccatgaggtggcaagaaatgggctacattttctaccccagaaaactacgatagcccttatgaaacttaagggtcgaaggtggatttagcagtaaactgagagtagagtgcttagttgaacagggccctgaagcgcgtacacaccgcccgtcaccctcctcaagtatacttcaaaggacatttaactaaaacccctacgcatttatatagaggagacaagtcgtaacatggtaagtgtactggaaagtgcacttggacgaaccagagtgtagcttaacacaaagcacccaacttacacttaggagatttcaacttaacttgaccgctctgagctaaacctagccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacaatcaaccaacaagtcattattaccctcactgtcaacccaacacaggcatgctcataaggaaaggttaaaaaaagtaaaaggaactcggcaaatcttaccccgcctgtttaccaaaaacatcacctctagcatcaccagtattagaggcaccgcctgcccagtgacacatgtttaacggccgcggtaccctaaccgtgcaaaggtagcataatcacttgttccttaaatagggacctgtatgaatggctccacgagggttcagctgtctcttacttttaaccagtgaaattgacctgcccgtgaagaggcgggcatgacacagcaagacgagaagaccctatggagctttaatttattaatgcaaacagtacctaacaaacccacaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccgatggtgcagccgctattaaaggttcgtttgntcaacgattaaagtcctacgtgatctgagttcagaccggagtaatccaggtcggtttctatctacttcaaattcctccctgtacgaaaggacaagagaaataaggcctacttcacaaagcgccttcccccgtaaatgatatcatctcaacttagtattatacccacacccacccaagaacagggtttgttaagatggcagagcccggtaatcgcataaaacttaaaactttacagtcagaggttcaattcctcttcttaacaacatacccatggccaacctcctactcctcattgtacccattctaatcgcaatggcattcctaatgcttaccgaacgaaaaattctaggctatatacaactacgcaaaggccccaacgttgtaggcccctacgggctactacaacccttcgctgacgccataaaactcttcaccaaagagcccctaaaacccgccacatctaccatcaccctctacatcaccgccccgaccttagctctcaccatcgctcttctactatgaacccccctccccatacccaaccccctggtcaacctcaacctaggcctcctatttattctagccacctctagcctagccgtttactcaatcctctgatcagggtgagcatcaaactcaaactacgccctgatcggcgcactgcgagcagtagcccaaacaatctcatatgaagtcaccctagccatcattctactatcaacattactaataagtggctcctttaacctctccacccttatcacaacacaagaacacctctgattactcctgccatcatgacccttggccataatatgatttatctccacactagcagagaccaaccgaacccccttcgaccttgccgaaggggagtccgaactagtctcaggcttcaacatcgaatacgccgcaggccccttcgccttattcttcatagccgaatacacaaacattattataataaacaccctcaccactacaatcttcctaggaacaacatatgacgcactctcccctgaactctacacaacatattttgttaccaagaccctacttctaacctccctgttcttatgaattcgaacagcatacccccgattccgctacgaccaactcatacacctcctatgaaaaaacttcctaccactcaccctagcattacttatatgatatgtttccatacccattacaatctccagcattccccctcaaacctaagaaatatgtctgataaaagagttactttgatagagtaaataataggagcttaaacccccttatttctaggactatgagaatcgaacccatccctgagaatccaaaattctccgtgccacctatcacaccccatcctaaagtaaggtcagctaaataagctatcgggcccataccccgaaaatgttggttatacccttcccgtactaattaatcccctggcccaacccgtcatctactctaccatctttgcaggcacactcatcacagcgctaagctcgcactgattttttacctgagtaggcctagaaataaacatgctagcttttattccagttctaaccaaaaaaataaaccctcgttccacagaagctgccatcaagtatttcctcacgcaagcaaccgcatccataatccttctaatagctatcctcttcaacaatatactctccggacaatgaaccataaccaatactaccaatcaatactcatcattaataatcataatggctatagcaataaaactaggaatagccccctttcacttctgagtcccagaggttacccaaggcacccctctgacatccggcctgcttcttctcacatgacaaaaactagcccccatctcaatcatataccaaatctctccctcactaaacgtaagccttctcctcactctctcaatcttatccatcatagcaggcagttgaggtggattaaaccaaacccagctacgcaaaatcttagcatactcctcaattacccacataggatgaataatagcagttctaccgtacaaccctaacataaccattcttaatttaactatttatattatcctaactactaccgcattcctactactcaacttaaactccagcaccacgaccctactactatctcgcacctgaaacaagctaacatgactaacacccttaattccatccaccctcctctccctaggaggcctgcccccgctaaccggctttttgcccaaatgggccattatcgaagaattcacaaaaaacaatagcctcatcatccccaccatcatagccaccatcaccctccttaacctctacttctacctacgcctaatctactccacctcaatcacactactccccatatctaacaacgtaaaaataaaatgacagtttgaacatacaaaacccaccccattcctccccacactcatcgcccttaccacgctactcctacctatctccccttttatactaataatcttatagaaatttaggttaaatacagaccaagagccttcaaagccctcagtaagttgcaatacttaatttctgtaacagctaaggactgcaaaaccccactctgcatcaactgaacgcaaatcagccactttaattaagctaagcccttactagaccaatgggacttaaacccacaaacacttagttaacagctaagcaccctaatcaactggcttcaatctacttctcccgccgccgggaaaaaaggcgggagaagccccggcaggtttgaagctgcttcttcgaatttgcaattcaatatgaaaatcacctcggagctggtaaaaagaggcctaacccctgtctttagatttacagtccaatgcttcactcagccattttacctcacccccactgatgttcgccgaccgttgactattctctacaaaccacaaagacattggaacactatacctattattcggcgcatgagctggagtcctaggcacagctctaagcctccttattcgagccgagctgggtcagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgtagacctaaccatcttctccttacacctagcaggtgtctcctctatcttaggggccatcaacttcatcacaacaattatcaatataaaaccccctgccataacccaataccaaacgcccctcttcgtctgatccgtcctaatcacagcagtcctacttctcctatctctcccagtcctagctgctggcatcactatactactaacagaccgcaacctcaacaccaccttcttcgaccccgccggaggaggagaccccattctataccaacacctattctgatttttcggtcaccctgaagtttatattcttatcctaccaggcttcggaataatctcccatattgtaacttactactccggaaaaaaagaaccatttggatacataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctaactggcattgtattagcaaactcatcactagacatcgtactacacgacacgtactacgttgtagctcacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatcctatcatctgtaggctcattcatttctctaacagcagtaatattaataattttcatgatttgagaagccttcgcttcgaagcgaaaagtcctaatagtagaagaaccctccataaacctggagtgactatatggatgccccccaccctaccacacattcgaagaacccgtatacataaaatctagacaaaaaaggaaggaatcgaaccccccaaagctggtttcaagccaaccccatggcctccatgactttttcaaaaaggtattagaaaaaccatttcataactttgtcaaagttaaattataggctaaatcctatatatcttaatggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaaccaggcgacctgcgactccttgacgttgacaatcgagtagtactcccgattgaagcccccattcgtataataattacatcacaagacgtcttgcactcatgagctgtccccacattaggcttaaaaacagatgcaattcccggacgtctaaaccaaaccactttcaccgttacacggccgggggtatactacggtcaatgctctgaaatctgtggagcaaaccacagtttcatgcccatcgtcctagaattaattcccctaaaaatctttgaaatagggcccgtatttaccctatagcaccccctctaccccccctagagcccactgtaaagctaacttagcattaaccttttaagttaaagattaagagaaccaacacctctttacagtgaaatgccccaactaaatactaccgtatggcccaccataattacccccatactccttacactattcctcatcacccaactaaaaatattaaacacaaactaccacctacctccctcaccaaagcccataaaaataaaaaattataacaaaccctgagaaccaaaatgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcgcagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaaacgccaccctagcaatatcaaccattaaccttccctctacacttatcatcttcacaattctaattctactgactatcctagaaatcgctgtcgccttaatccaagcctacgttttcacacttctagtaagcctctacctgcacgacaacacataatgacccaccaatcacatgcctatcatatagtaaaacccagcccatgacccctaacaggggccctctcagccctcctaatgacctccggcctagccatgtgatttcacttccactccataacgctcctcatactaggcctactaaccaacacactaaccatataccaatgatggcgcgatgtaacacgagaaagcacataccaaggccaccacacaccacctgtccaaaaaggccttcgatacgggataatcctatttattacctcagaagtttttttcttcgcaggatttttctgagccttttaccactccagcctagcccctaccccccaattaggaggacactggcccccaacaggcatcaccccgctaaatcccctagaagtcccactcctaaacacatccgtattactcgcatcaggagtatcaatcacctgagctcaccatagtctaatagaaaacaaccgaaaccaaataattcaagcactgcttattacaattttactgggtctctattttaccctcctacaagcctcagagtacttcgagtctcccttcaccatttccgacggcatctacggctcaacattttttgtagccacaggcttccacggacttcacgtcattattggctcaactttcctcactatctgcttcatccgccaactaatatttcactttacatccaaacatcactttggcttcgaagccgccgcctgatactggcattttgtagatgtggtttgactatttctgtatgtctccatctattgatgagggtcttactcttttagtataaatagtaccgttaacttccaattaactagttttgacaacattcaaaaaagagtaataaacttcgccttaattttaataatcaacaccctcctagccttactactaataattattacattttgactaccacaactcaacggctacatagaaaaatccaccccttacgagtgcggcttcgaccctatatcccccgcccgcgtccctttctccataaaattcttcttagtagctattaccttcttattatttgatctagaaattgccctccttttacccctaccatgagccctacaaacaactaacctaccactaatagttatgtcatccctcttattaatcatcatcctagccctaagtctggcctatgagtgactacaaaaaggattagactgaaccgaattggtatatagtttaaacaaaacgaatgatttcgactcattaaattatgataatcatatttaccaaatgcccctcatttacataaatattatactagcatttaccatctcacttctaggaatactagtatatcgctcacacctcatatcctccctactatgcctagaaggaataatactatcgctgttcattatagctactctcacaaccctcaacacccactccctcttagccaatattgtgcctattgccatactagtctttgccgcctgcgaagcagcggtgggcctagccctactagtctcaatctccaacacatatggcctagactacgtacataacctaaacctactccaatgctaaaactaatcgtcccaacaattatattactaccactgacatgactttccaaaaaacacataatttgaatcaacacaaccacccacagcctaattattagcatcatccctctactattttttaaccaaatcaacaacaacctatttagctgttccccaaccttttcctccgaccccctaacaacccccctcctaatactaactacctgactcctacccctcacaatcatggcaagccaacgccacttatccagtgaaccactatcacgaaaaaaactctacctctctatactaatctccctacaaatctccttaattataacattcacagccacagaactaatcatattttatatcttcttcgaaaccacacttatccccaccttggctatcatcacccgatgaggcaaccagccagaacgcctgaacgcaggcacatacttcctattctataccctagtaggctcccttcccctactcatcgcactaatttacactcacaacaccctaggctcactaaacattctactactcactctcactgcccaagaactatcaaactcctgagccaacaacttaatatgactagcttacacaatagcttttatagtaaagatacctctttacggactccacttatgactccctaaagcccatgtcgaagcccccatcgctgggtcaatagtacttgccgcagtactcttaaaactaggcggctatggtataatacgcctcacactcattctcaaccccctgacaaaacacatagcctaccccttccttgtactatccctatgaggcataattataacaagctccatctgcctacgacaaacagacctaaaatcgctcattgcatactcttcaatcagccacatagccctcgtagtaacagccattctcatccaaaccccctgaagcttcaccggcgcagtcattctcataatcgcccacggacttacatcctcattactattctgcctagcaaactcaaactacgaacgcactcacagtcgcatcataatcctctctcaaggacttcaaactctactcccactaatagctttttgatgacttctagcaagcctcgctaacctcgccttaccccccactattaacctactgggagaactctctgtgctagtaaccacgttctcctgatcaaatatcactctcctacttacaggactcaacatactagtcacagccctatactccctctacatatttaccacaacacaatggggctcactcacccaccacattaacaacataaaaccctcattcacacgagaaaacaccctcatgttcatacacctatcccccattctcctcctatccctcaaccccgacatcattaccgggttttcctcttgtaaatatagtttaaccaaaacatcagattgtgaatctgacaacagaggcttacgaccccttatttaccgagaaagctcacaagaactgctaactcatgcccccatgtctaacaacatggctttctcaacttttaaaggataacagctatccattggtcttaggccccaaaaattttggtgcaactccaaataaaagtaataaccatgcacactactataaccaccctaaccctgacttccctaattccccccatccttaccaccctcattaaccctaacaaaaaaaactcatacccccattatgtaaaatccattgtcgcatccacctttattatcagtctcttccccacaacaatattcatgtgcctagaccaagaagttattatctcgaactgacactgagccacaacccaaacaacccagctctccctaagcttcaaactagactacttctccataatattcatccctgtagcattgttcgttacatggtccatcatagaattctcactgtgatatataaactcagacccaaacattaatcagttcttcaaatatctactcatcttcctaattaccatactaatcttagttaccgctaacaacctattccaactgttcatcggctgagagggcgtaggaattatatccttcttgctcatcagttgatgatacgcccgagcagatgccaacacagcagccattcaagcaatcctatacaaccgtatcggcgatatcggttttatcctcgccttagcatgatttatcctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccacatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctaccatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataacctattcccccgagcaatctcaattacaatatatacaccaacaaacaatgttcaaccagtaactactactaatcaacgcccataatcatacaaagcccccgcaccaataggatcctcccgaatcaaccctgacccctctccttcataaattattcagcttcctacactattaaagtttaccacaaccaccaccccatcatactctttcacccacagcaccaatcctacctccatcgctaaccccactaaaacactcaccaagacctcaacccctgacccccatgcctcaggatactcctcaatagccatcgctgtagtatatccaaagacaaccatcattccccctaaataaattaaaaaaactattaaacccatataacctcccccaaaattcagaataataacacacccgaccacaccgctaacaatcaatactaaacccccataaataggagaaggcttagaagaaaaccccacaaaccccattactaaacccacactcaacagaaacaaagcatacatcattattctcgcacggactacaaccacgaccaatgatatgaaaaaccatcgttgtatttcaactacaagaacaccaatgaccccaatacgcaaaattaaccccctaataaaattaattaaccactcattcatcgacctccccaccccatccaacatctccgcatgatgaaacttcggctcactccttggcgcctgcctgatcctccaaatcaccacaggactattcctagccatgcactactcaccagacgcctcaaccgccttttcatcaatcgcccacatcactcgagacgtaaattatggctgaatcatccgctaccttcacgccaatggcgcctcaatattctttatctgcctcttcctacacatcgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgattctttacctttcacttcatcttgcccttcattattgcagccctagcagcactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcctgtccttgtagtataaactaatacaccagtcttgtaaaccggagatgaaaacctttttccaaggacaaatcagagaaaaagtctttaactccaccattagcacccaaagctaagattctaatttaaactattctctgttctttcatggggaagcagatttgggtaccacccaagtattgactcacccatcaacaaccgctatgtatttcgtacattactgctagccaccatgaatattgtacagtaccataaatacttgaccacctgtagtacatgaaaacccaacccacatcaaaaccccctccccatgcttacaagcaagtacagcaatcaaccctcaactatcacacatcaactgcaactccaaagccacccctcacccactaggataccaacaaacctacccacccttaacagcacatagtacataaagccatttaccgtacatagcacattacagtcaaatcccttctcgtccccatggatgacccccctcagataggggtcccttgaccaccatcctccgtgaaatcaatatcccgcacaagagtgctactctcctcgctccgggcccataacacttgggggtagctaaagtgaactgtatccgacatctggttcctacttcagggccataaagcctaaatagcccacacgttccccttaaataagacatcacgatg

SEQ ID NO: 19 - DNA, 23 bp, Homo sapiens
cccctacgcatttatatagagga

SEQ ID NO: 20 - DNA, 23 bp, Homo sapiens
cgtccaagtgcactttccagtac

SEQ ID NO: 21 - DNA, 18 bp, Homo sapiens
gaggagacaagtcgtaac

SEQ ID NO: 22 - DNA, 17 bp, Homo sapiens
ttacgacttgcctcctc

SEQ ID NO: 23 - DNA, 20 bp, Homo sapiens
cccaccctaccacacattcg

SEQ ID NO: 24 - DNA, 19 bp, Homo sapiens
gggggttcgattccttcct

SEQ ID NO: 25 - DNA, 18 bp, Homo sapiens
cttccttttttgtctaga

SEQ ID NO: 26 - DNA, 17 bp, Homo sapiens
ttccttttttggctaga

SEQ ID NO: 27 - DNA, 264 bp, Cytomegalovirus
tttgttgtaaatggccgagagaatggctgacgggtgatctttgctgagttccttgaagacctctagggtgcgccgttgatccacacaccaggcttctgcgatttgcgccagcgcccggttgatgtaaccgcgcaacgtgtcataggtgaactgcagctgggcgtagaccagattgtgcaccgattccatgctggacaaatgagttgtattattgtcactcgtacttcttctggtcctatgagtgatattcagactggatcgatt

SEQ ID NO: 28 - DNA, 22 bp, Cytomegalovirus
tttgttgtaaatggccgagaga

SEQ ID NO: 29 - DNA, 18 bp, Cytomegalovirus
caacggcgcaccctagag

SEQ ID NO: 30 - DNA, 18 bp, Cytomegalovirus
atcttgctgagttccttg